(19) United States
(12) Patent Application Publication
(10) Pub. No.: US 2023/0117968 A1
(43) Pub. Date: Apr. 20, 2023

(54) NEBULIZER SYSTEM FOR A MOTOR VEHICLE

(71) Applicant: VALEO SYSTEMES THERMIQUES, La Verriere (FR)

(72) Inventors: Vincent FEUILLARD, La Verriere (FR); Julien MIKHEL, La Verriere (FR); Clement ESCULIER, La Verriere (FR); Yves ROUSSEAU, La Verriere (FR)

(73) Assignee: VALEO SYSTEMES THERMIQUES, La Verriere (FR)

(21) Appl. No.: 17/796,136

(22) PCT Filed: Jan. 28, 2021
(86) PCT No.: PCT/FR2021/050155

§ 371 Date: Jul. 28, 2022

(30) Foreign Application Priority Data
Jan. 29, 2020 (FR) 2000857

(51) Int. Cl. B60H 1/32; C02F 1/32

(52) U.S. Cl. CPC B60H 1/3202; C02F 1/325; C02F 2303/04; C02F 2103/023

(57) ABSTRACT

The invention relates to a nebulizer system for a motor vehicle, including at least one tank for storing a liquid, a nebulizer chamber, the nebulizer chamber having a nebulizer component provided with an acoustic wave emission device which is configured such that the liquid forms a mist of droplets of the liquid, the mist being intended to enter a passenger compartment of the motor vehicle, a light-emitting device configured to emit radiation in the ultraviolet spectrum, and a carrier of the light-emitting device, the at least one tank for storing the liquid being configured such that the carrier of the light-emitting device crosses the tank.

### CROSS-REFERENCE TO RELATED APPLICATIONS

This application is filed under 35 U.S.C. § 371 U.S. National Phase of International Application No. PCT/FR2021/050155 filed Jan. 28, 2021 (published as WO2021152260), which claims priority benefit to French application No. 2000857 filed on Jan. 29, 2020, the disclosures of which are herein incorporated by reference in their entirety.

### TECHNICAL FIELD

The subject of the invention is a nebulizer system for a motor vehicle.

### BACKGROUND OF THE INVENTION

In a known manner, a nebulizer system comprises a reservoir for storing a liquid of liquid water and a device for emitting acoustic waves configured such that the water coming from the reservoir for storing a liquid forms a mist of water droplets.

Such a nebulizer system makes it possible to cool an air flow in which the mist of water is sprayed.

In a motor vehicle, even one provided with a ventilation and/or air-conditioning device, it is not uncommon for the users of the vehicle to suffer under heat in the vehicle interior, in particular the rear seat passengers, who are farther away from the air vents of the ventilation and/or air-conditioning device.

As a result, it is advantageous to make use of a nebulizer system, since the mist of water rapidly cools the air in the vehicle interior, providing a sensation of immediate cold while still rehumidifying it.

Because the water coming from the nebulizer system comes in direct contact with the skin of the users of the vehicle, it is essential to ensure that the nebulizer system is hygienic.

For the other part, the nebulizer system must be as compact as possible because it constitutes an additional element of the vehicle that it occupies.

The objective of the invention is to propose a nebulizer system that is not dangerous to the health of the users of the motor vehicle while still being compact.

### BRIEF SUMMARY OF THE INVENTION

To that end, a subject of the invention is a nebulizer system for a motor vehicle, comprising at least one reservoir for storing a liquid, a nebulizer enclosure, said nebulizer enclosure comprising a nebulizing component provided with a device for emitting acoustic waves configured such that said liquid forms a mist of droplets of said liquid, the mist being intended to enter an interior of the motor vehicle, an electroluminescent device configured to emit radiation in the ultraviolet spectrum, and a support for said electroluminescent device, said at least one reservoir for storing a liquid being configured so as to be traversed by the support of the electroluminescent device.

Thus, by virtue of the electroluminescent device, the bacteria that are able to proliferate in the liquid circulating in the nebulizer system are effectively neutralized, without it being necessary to make use of a filter, the very shape of the support ensuring that the nebulizer system is more compact.

According to another feature of the invention, the ultraviolet spectrum comprises wavelengths in a range of between 200 nm and 280 nm.

According to another feature of the invention, said at least one reservoir for storing a liquid comprises a through-orifice for the electroluminescent device.

According to another feature of the invention, said at least one reservoir for storing a liquid comprises a through-well forming a housing for the support of the electroluminescent device.

According to another feature of the invention, the support for the electroluminescent device comprises a duct configured to be arranged in the housing for said at least one reservoir for storing a liquid.

According to another feature of the invention, the duct delimits a hollow internal space so as to ensure a supply of electrical power to the electroluminescent device.

According to another feature of the invention, said at least one reservoir for storing a liquid comprises a wall on which said electroluminescent device rests.

According to another feature of the invention, the electroluminescent device is oriented so as to irradiate the mist of droplets in the nebulizer enclosure.

According to another feature of the invention, the system comprises a heat sink associated with the electroluminescent device.

According to another feature of the invention, the heat sink comprises a cavity for housing the support for the electroluminescent device.

Another subject of the invention is a ventilation, heating and/or air conditioning device for a motor vehicle, comprising a nebulizer system as described above.

### BRIEF DESCRIPTION OF THE DRAWINGS

Other features, details and advantages of the invention will become apparent upon reading the detailed description below, and upon analyzing the appended drawings, in which:

FIG. 1 illustrates a partial front view of a nebulizer system according to the present invention, and

FIG. 2 illustrates a partial perspective view in section of the system of FIG. 1 according to another embodiment.

### DETAILED DESCRIPTION OF THE INVENTION

A subject of the invention is a nebulizer system 10 for a motor vehicle.

The nebulizer system 10 makes it possible to cool an air flow intended for the interior of the motor vehicle, as will be described in more detail. The nebulizer system 10 is intended to be incorporated in a central console of the vehicle, in particular.

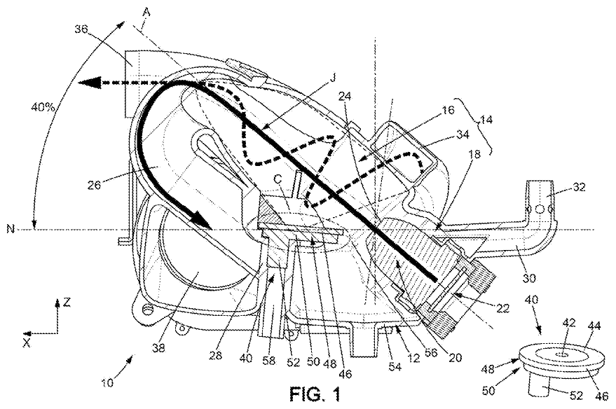

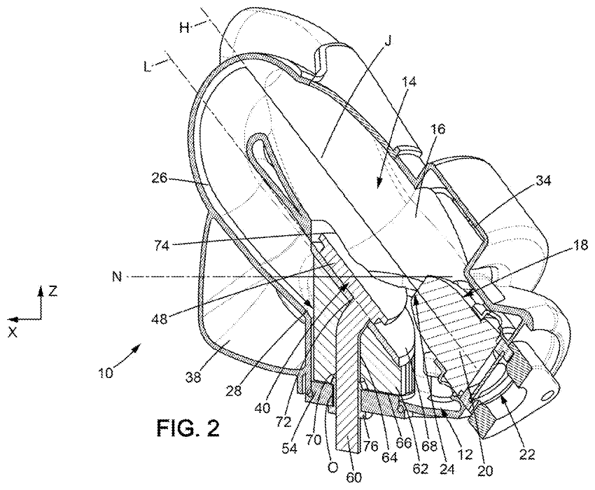

The nebulizer system 10 is described in accordance with the specific configuration of FIGS. 1 and 2, but it will be clearly understood that the invention is not limited to these embodiments.

As can be seen in the figures, the nebulizer system 10 comprises a reservoir 12 for storing a fluid, preferably liquid water, otherwise referred to as tank.

The nebulizer system 10 also comprises a nebulizer enclosure 14. Here, the nebulizer enclosure 14 is realized in the form of an elongate hollow body extending along a longitudinal axis A.

In the embodiment illustrated in the figures, the longitudinal axis A is inclined with respect to a vertical direction Z and with respect to a horizontal direction X.

As is apparent in particular from the figures, the nebulizer enclosure 14 has at least two parts arranged one after the other in the direction of the longitudinal axis A, specifically a nebulizer chamber 16 and a nebulizer tube 18, the nebulizer tube 18 leading into the nebulizer chamber 16.

As is apparent from FIGS. 1 and 2, the nebulizer tube 18 has a nebulizing component realized here in the form of a nebulizer nozzle 20 provided with a device for emitting acoustic waves transmitted into the liquid, which device is configured such that the surface of the liquid coming from the reservoir 12 for storing the liquid generates a mist of droplets of the liquid, this mist being intended to enter an interior of the motor vehicle.

It will be noted that the longitudinal axis A also corresponds to the direction in which the jet J of water from which the mist detaches is ejected from the nebulizer nozzle 20 into the nebulizer chamber 16.

As can be seen in the figures, the longitudinal axis of the nebulizer nozzle 20 coincides with the longitudinal axis A of the nebulizer enclosure 14.

The nebulizer nozzle 20 is arranged at least partially inside the nebulizer enclosure 14. The nebulizer nozzle 20 has a lateral wall delimiting an interior volume which is able to contain the liquid to be nebulized. The internal cross section of this lateral wall narrows gradually in the direction of an outlet orifice 24 for the liquid, making it possible to form an acoustic wave concentrator.

A piezoelectric (ceramic) element 22 is arranged on the opposite side to an outlet orifice 24 for the liquid.

The piezoelectric element 22 is able to emit acoustic waves into the liquid to be sprayed, thereby making it possible to generate a mist of liquid droplets when the nebulizer nozzle 20 is filled by the latter and when the piezoelectric element 22 emits acoustic waves of a suitable frequency and intensity. The piezoelectric element 22 is preferably able to emit ultrasound at a frequency of between 1 MHz and 3 MHz, in particular between 1.7 MHz and 2.4 MHz.

For example, the diameter of the droplets present in the mist is less than 10 μm.

The nebulizer nozzle 20 also has at least one intake orifice for the liquid to be nebulized which allows the introduction of the liquid to be nebulized into the interior volume of the nebulizer nozzle 20, in fluidic communication with the reservoir 12 for storing the liquid.

As is apparent in FIGS. 1 and 2, the nebulizer system 10 also comprises a return duct 26 for returning the liquid to the reservoir 12 for storing the liquid. The return duct 26 for returning the liquid continues the nebulizer chamber 16 with formation of an almost 180° curve as far as an end 28. The curved shape of the return duct 26 for returning the liquid ensures improved compactness of the nebulizer system 10.

The nebulizer system 10 also comprises an inlet channel 30 for water provided with an end piece 32 intended to accommodate a cartridge that contains liquid and thereby constitutes an additional, advantageously removable reservoir, for storing the liquid, of the nebulizer system 10, as illustrated in FIG. 1.

The nebulizer system 10 further has an inlet duct 34 for air and also at least one outlet duct 36 for the mist, a fan 38 forcing the movement of the air.

Thus, the air enters the nebulizer enclosure 14 at the nebulizer chamber 16, where the air and the nebulized liquid mix before the mixture leaves the nebulizer system 10 via the outlet duct 36, where it circulates to the interior of the vehicle.

The nebulizer system 10 also comprises an electroluminescent device 40, illustrated in FIGS. 1 and 2.

The electroluminescent device 40 has an ultraviolet radiation source 42 with emitted wavelengths of between 200 nm and 280 nm (this is ultraviolet C radiation). The proportion of effective radiation, i.e. the proportion of radiation which makes it possible to kill the bacteria, is between 200 and 248 nm. However, the radiation source can emit wavelengths of between 248 and 400 nm, even if the performance capabilities are lower in terms of the proportion of bacteria killed.

Advantageously, the electroluminescent device 40 consists of a photolysis reactor with light-emitting diode(s) emitting wavelengths of between between 200 and 280 nm. Light-emitting diodes are ultraviolet radiation sources with numerous advantages. They have low power consumption, operate with DC supply voltage and do not cause electromagnetic compatibility problems, thereby facilitating their integration within the nebulizer system 10. They also have small dimensions, thereby making it possible to maintain a compact system. In addition, they do not require the use of mercury and have a long service life, which is an advantage in terms of recycling and maintenance problems.

The ultraviolet radiation source 42, and also an associated electronic circuit, are embedded in a resin 44 bordered by a seal 46. The electroluminescent device 40 comprises a support structure 48, constituted by a base 50, preferably made of stainless steel, arranged under the resin 44 and, advantageously, a support duct 52, for example perpendicular to the base 50.

As is apparent in the figures, the reservoir 12 for storing the liquid (otherwise referred to as tank) is configured so as to be traversed by the support structure 48.

The first embodiment, illustrated in FIG. 1, will now be described in detail.

In FIG. 1, the reservoir 12 comprises a planar base wall 54 and an upper wall 56 facing the base wall 30. The electroluminescent device 40 is arranged on the upper wall 56 such that, during operation, the base 50 is submerged in the water whereas the resin 44 is arranged above the water level. The submersion of the base 50 ensures effective cooling of the electroluminescent device 40.

The reservoir 12 comprises a well 58 for housing the support duct 52 of the electroluminescent device 40 passing through the reservoir 12 between two hollow ends, one of which is in the base wall 54, the other of which is in the upper wall 56. The well 58 provides leak tightness, in order in particular to supply electrical power to the electroluminescent device 40 in complete safety. The well 58 also ensures greater compactness of the nebulizer system 10.

In FIG. 1, the base 50 is arranged parallel to the water level N in the reservoir 12, the ultraviolet radiation source 42 being oriented toward the nebulizer chamber 16. Thus, the radiation emitted by the ultraviolet radiation source 42 delimits a cone C which irradiates the jet of mist flowing in the nebulizer chamber 16. This configuration has the advantage of directly treating the air, which is where bacteria particularly proliferate.

It will be noted that during operation, the cartridge containing liquid is secured to the dedicated end piece, and the water level N reaches the duct, the nebulizer tube 18, the end of the return duct 26, and the upper wall 56, with the well 58 being surrounded by water.

It will also be noted that this configuration makes it possible not to equip the nebulizer system 10 with a water circulation pump.

The second embodiment, illustrated in FIG. 2, will now be described in detail.

In FIG. 2, the reservoir 12 comprises a planar base wall 54 comprising a through-orifice O for the support channel 60, as will be described in detail.

As can also be seen in FIG. 2, the nebulizer system 10 comprises a heat sink 62 arranged between the return duct 26 and the nebulizer nozzle 20. A cavity 64 for housing the support channel 60 of the electroluminescent device 40, passing through between a first orifice 70 in a first wall, referred to as bottom wall 66, and a second orifice 72 in a second wall, referred to as top wall 68, is made in the heat sink 62.

The bottom wall 66 rests on the base wall 54 of the reservoir 12, whereas the top wall 68 faces a wall portion 74 of the nebulizer chamber 16.

As illustrated in FIG. 2, the first orifice 70 in the bottom wall 66 is arranged facing the orifice O in the base wall 54, a gland 76 providing leak tightness between the orifice O in the base wall 54 and the first orifice 70 in the bottom wall 66.

The top wall 68 extends in a direction L which is inclined relative to the X and Z axes, for example by 40° relative to the X axis. Advantageously, this inclination corresponds to the direction of the wall portion 74, as well as to the direction A of ejection of the mist J.

This parallel configuration of the direction L and the axis A allows the nebulizer chamber 16 to have a more circular section than the first embodiment, thereby improving the turbulence of the air in the nebulizer chamber 16 and optimizing the transmission of the mist toward the interior of the vehicle.

Owing to this parallel configuration, the electroluminescent device 40 is arranged higher up than for the first embodiment, and is located at least partially above the water level N.

In FIG. 2, the ultraviolet radiation source 42 is oriented toward the nebulizer chamber 16. Thus, the radiation emitted by the ultraviolet radiation source 42 delimits a cone which irradiates the jet of mist flowing in the nebulizer chamber 16. This configuration has the advantage of directly treating the air, which is where bacteria particularly proliferate.

It will be noted that during operation, the cartridge containing liquid is secured to the dedicated end piece, and the water level N reaches the duct, the nebulizer tube 18, the end of the return duct 26, and the upper wall, with the heat sink 62 being submerged in the water.

If the base 50 is not submerged, the heat sink 62 ensures sufficient cooling of the electroluminescent device 40, in particular on account of its submersion.

It will also be noted that this configuration makes it possible not to equip the nebulizer system 10 with a water circulation pump.

By virtue of the antibacterial portion, bacteria cannot proliferate in the nebulizer system 10, thereby ensuring that the nebulizer system 10 is always hygienic, whether the nebulizer system 10 is operating or not.

What is claimed is:

1. A nebulizer system for a motor vehicle, comprising:
at least one reservoir for storing a liquid,
a nebulizer enclosure, said nebulizer enclosure including a nebulizing component including a device for emitting acoustic waves that is configured such that said liquid coming from the at least one reservoir for storing the liquid forms a mist of droplets of said liquid, the mist being intended to enter an interior of the motor vehicle,
an electroluminescent device configured to emit radiation in the ultraviolet spectrum, and
a base for said electroluminescent device,
said at least one reservoir for storing the liquid being configured so as to be traversed by the base of the electroluminescent device.

2. The nebulizer system as claimed in claim 1, wherein the ultraviolet spectrum includes wavelengths in a range of between 200 nm and 280 nm.

3. The nebulizer system as claimed in claim 1, wherein said at least one reservoir for storing the liquid includes a through orifice for the electroluminescent device.

4. The nebulizer system as claimed in claim 1, wherein said at least one reservoir for storing the liquid includes a well forming a housing for the base of the electroluminescent device.

5. The nebulizer system as claimed in claim 1, wherein said at least one reservoir for storing the liquid includes a wall on which said electroluminescent device rests.

6. The nebulizer system as claimed in claim 1, wherein the electroluminescent device is oriented so as to irradiate the mist of droplets in the nebulizer enclosure.

7. The nebulizer system as claimed in claim 1, further comprising a heat sink associated with the electroluminescent device.

8. The nebulizer system as claimed in claim 7, wherein the heat sink includes a cavity for housing a support channel of the electroluminescent device.

9. A ventilation, heating and/or air-conditioning device for a motor vehicle, comprising a nebulizer system for a motor vehicle, including:
at least one reservoir for storing a liquid,
a nebulizer enclosure, said nebulizer enclosure including a nebulizing component including a device for emitting acoustic waves that is configured such that said liquid coming from the at least one reservoir for storing the liquid forms a mist of droplets of said liquid, the mist being intended to enter an interior of the motor vehicle,
an electroluminescent device configured to emit radiation in the ultraviolet spectrum, and
a base for said electroluminescent device,
said at least one reservoir for storing the liquid being configured so as to be traversed by the base of the electroluminescent device.
